(19) United States
(12) Patent Application Publication
(10) Pub. No.: US 2021/0353627 A1
(43) Pub. Date: Nov. 18, 2021

(54) NOVEL QUINAZOLINE EGFR INHIBITORS

(71) Applicant: Spectrum Pharmaceuticals, Inc., Irvine, CA (US)

(72) Inventor: Prasad V. Chaturvedula, Cheshire, CT (US)

(21) Appl. No.: 17/278,397

(22) PCT Filed: Sep. 20, 2019
(86) PCT No.: PCT/US19/52181

§ 371 Date: Mar. 22, 2021

Related U.S. Application Data
(60) Provisional application No. 62/734,655, filed on Sep. 21, 2018.

(51) Int. Cl. A61K 31/517; C07D 401/14; A61K 31/537; A61K 47/68; A61K 47/54

(52) U.S. Cl. CPC A61K 31/517; C07D 401/14; A61K 47/545; A61K 47/6849; A61K 31/537

(57) ABSTRACT

This document discloses a novel class of quinazoline EGFR inhibitors. Also disclosed are pharmaceutical compositions thereof and method for treating cancers. Disclosed herein is a novel class of quinazoline compounds which selectively and effectively inhibit the growth of cancer cells induced by the overexpression of an epidermal growth factor receptor (EGFR).

### CROSS-REFERENCE TO RELATED APPLICATIONS

This application claims the benefit of U.S. Provisional Application No. 62/734,655, filed on Sep. 21, 2018, the entire disclosures of which is incorporated herein by reference.

### TECHNICAL FIELD

Disclosed herein is a novel class of quinazoline compounds which selectively and effectively inhibit the growth of cancer cells induced by the overexpression of an epidermal growth factor receptor (EGFR).

### BACKGROUND

Approximately 10-12% of EGFR mutant NSCLC tumors have an in-frame insertion within exon 20 of EGFR (Arcila et al, 2012), and are generally resistant to EGFR TKIs. In addition, 90% of HER2 mutations in NSCLC are exon 20 mutations (Mazieres et al, 2013). Together, EGFR and HER2 exon 20 mutations comprise approximately 4% of NSCLC patients. The available TKIs of EGFR/HER2 (afatinib, lapatinib, neratinib, dacomitinib) have limited activity in patients with HER2 mutant tumors with many studies reporting OR rates below 40% (Kosaka et al, 2017), although some preclinical activity is observed in HER2 mouse models treated with afatinib (Perera et al, 2009; Robichaux et al, 2018). Recent studies on efficacy of poziotinib indicate its unique selectivity for EGFR and

HER 2 activating mutations at exon 19 or exon 20. Robichaux et al, have shown that poziotinib is a potent inhibitor of EGFR and HER2 with exon 20 isertion mutations (Robichaux et al, 2018). The authors hypothesized that poziotinib can effectively bind and inhibit EFGR drug binding pocket despite the configurational alteration caused by the exon 20 insertion mutation. Further testing revealed that poziotinib tightly binds deep into the sterically hindered drug binding pocket of EGFR with exon 20 insertion mutantion overcoming structural changes induced by exon 20 insertions. (Rochicaux et al, 2018).

Despite poziotinib's improved target selectivity to drug binding pocket of EGFR/HER 2 with exon 20 insertion mutation, there is an urgent need for new drugs to improve upon the low rate of survival among patients who may have resistance towards tyrosine kinase inhibitors due to presence of exon 19 or 20 mutation in EGFR/HER 2. The present invention discloses compounds that help overcome drug resistance in such cancer patient population.

### SUMMARY OF THE INVENTION

The compounds disclosed herein exhibit potent EGFR inhibitive activities particularly for patients who are resistant to conventional drug treatment or are at risk of developing drug resistance caused by the mutation of EGFR tyrosine kinase. In at least one embodiment, the mutation may include EGFR and/or HER2 exon 20 insertion mutation. In another embodiment, the mutation may include EFGR exon 19 deletion.

Compounds of the present invention have a better adverse event profile than other EGFR inhibitors. For instance, serious adverse events such as rash and diarrhea from convential EGFR inhibitors can be reduced with the compounds described herein. An aspect of the disclosure provide a compound or a pharmaceutical salt thereof, wherein the compound is represented by Formula I.

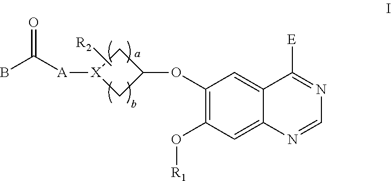

R₁ is hydrogen, C₁₋₆ alkyl, or C₁₋₆ alkyl substituted with C₁₋₆ alkoxy or 5- or 6-membered heterocyclic group having at feast one selected from the group consisting of N, O and S; R₂ is hydrogen, —COOH, C₁₋₆ alkyloxycarbonyl, or amido N-unsubstifuted or N-substituted with Y;
Y is hydroxy or C₁₋₆ alkyl or C₁₋₆ afkyl substituted with Z;
Z is hydroxy, C₁₋₆ alkoxy, C₁₋₆alkylthio, C₁₋₃ alkyfsuffonyl, di-C₁₋₃salkylaroine, C₁₋₆ alkyl, phenyl or 5- or 6-membered aromatic or non-aromatic heterocyclic group, said heterocyclic group containing one to four of the moiety selected from the group consisting of N, O, S, SO, and SO₂ and said aryl and heterocyclic group being unsubstituted, or substituted with substituents selected from the group consisting of halogen, hydroxyl, amino, nitro, cyano, C₁₋₆ alkyl, C₂₋₆ alkenyl, C₂₋₆ alkynyl, C₁₋₆ alkoxy, C₁₋₆ monoalkylamino and C₁₋₆ dialkylamino. A is NH, or NC₁₋₆ alkyl when X is CH; alternatively, A is void when X is N or NH;
B is

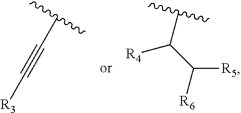

wherein:

R₁, R₄, R₅ and R₆ are each independently selected from the group consisting of hydrogen, halogen, N—C₁₋₆ alkylor N-hydroxy amidoor C—C alkyl reverse amido(—NHCOC₁₋₆), hydroxycarbonyl (—COOH), C₁₋₆ alkyloxycarbonyl (—COOC₁₋₆). C₁₋₆ alkyl, and C₁₋₆ alkyl substituted with a hydroxy, di-C₁₋₆ alkylamine or 3 to 6-membered heterocyclic group having at least one selected from the group consisting of N, O and S, wherein the 5- or 6-membered heterocyclic group is unsubstituled or substituted with C₁₋₄ alkyl: E is selected from the group consisting of

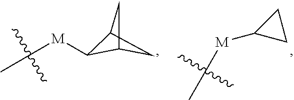

and 9 to 12 membered bicyclic ring,

wherein each of these is optionally substituted with one or more substituents selected from the group consisting of halogen, hydroxy, cyano, nitro, (mono-, di-, or trihalogeno)nethyl, mercapto, C₁₋₆ alkylthio, acrylamido, C₁₋₆ alkyl, C₂₋₆ alkenyl, C₂₋₆ alkynyl, C₁₋₆ alkoxy, phenyloxy, and C₁₋₆ dialkylamino, further wherein M is selected from the group consisting of O, S, NH, NC₁₋₆ alkyl and C₁₋₅ alkyl;
and
a and b are each an integer ranging from 0 to 6. In some embodiments, a and b are independently 1 or 2.

In some embodiments, E is

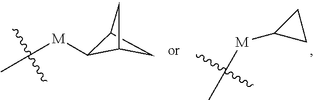

each of which is optionally substituted to one to three halogens; and M is NH.

In some embodiments, E is a 5,5-, 5,6- or 6,6-bicyclic ring system containing up to three hetero atoms selected from oxygen, nitrogen or sulfur.

In some embodiments, E is the bicyclic ring selected from the group consisting of naphthyridine, indole, benzoimidazole, benzotriazole, benzodioxaole, furopyridine, isoindole, pyridooxazine, pyrrolopyridine, quinoxaline, quinazoline, quinoline, isoquinoline, indazole, [1,2,4]triazolo [1,5-a]pyridine, 1,2,3,4-tetrahydroisoquinoline, 1,3-benzodioxole, 1-benzothiophene, 1H-indazole, 1H-pyrrolol[2,3-b]pyridine, 1H-pyrrolol[2,3-c]pyridine, 1H-pyrrolo[3,2-b]pyridine, 1H-pyrrolo[3,2-c]pyridine, 2,1,3-benzoxadiazole, 3,4-dihydro -2H-pyrido[3,2-b][1,4]oxazine, 3H-imidazo[4,5-b ][pyridine, 4,5,6,7]-tetrahydropyrazolo[1,5-a]pyridine, furo[2,3-c]pyridine, furo[3,2-b]pyridine, imidazo[1,2-a]pyridine, and thieno[3,2-c]pyridin-4(5H)-one; wherein each is optionally substituted with one or more substituents selected from the group consisting of halogen, hydroxy, cyano, nitro, (mono-, di-, or trihalogeno)methyl, mercapto, C₁₋₆ alkylthio, acrylamido, C₁₋₆ alkyl, C₂₋₆ alkenyl, C₂₋₆ alkynyl, C₁₋₆ alkoxy, phenyloxy, and C₁₋₆ dialkylamino.

In some embodiments, E is the optionally substituted bicyclic ring selected from the group consisting of naphthyridine, indole, benzoimidazole, benzotriazole, isoindole, quinoxaline, quinazoline, quinoline, isoquinoline, and indazole.

In some embodiments, E is the optionally substituted bicyclic ring selected from the group consisting of

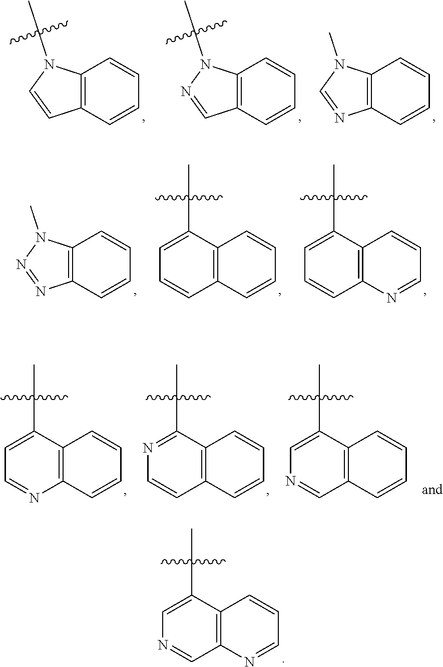

In some embodiments, the optional substituents include 1, 2 or 3 same or different halogens (e.g. F, Cl, Br, or I).

In some embodiments, E is selected from the group consisting of

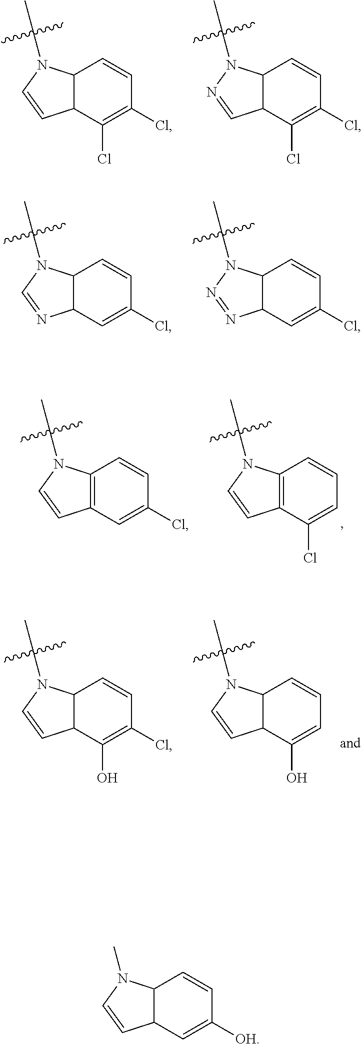

In some embodiments, E is a phenylamino (—NHPh), wherein the phenyl is substituted with a heteroary and optionally one or more substituents selected from the group consisting of halogen and C₁₋₆ alkyl. In some embodiments, the heteroary sustituent on phenyl is selected from the group consisiting of oxazole, thizaole, pyrrole, imidazole, and pyrazole. In some embodiments, E is a phenylamino (—NHPh), wherein the phenyl is substituted with oxazole and optionally one or more halogens.

In some embodiments, the moiety of

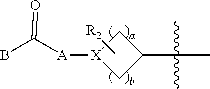

is selected from the group consisting of

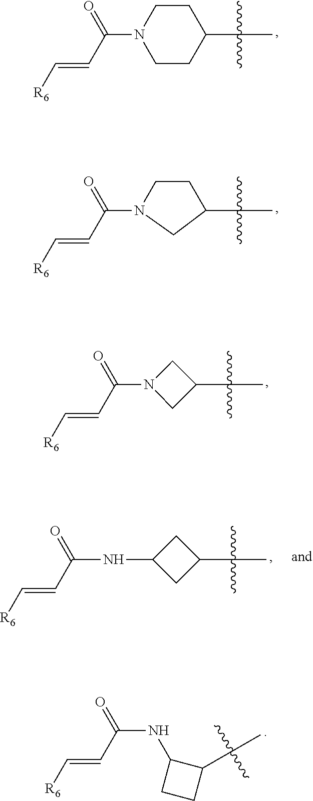

In some embodiments. R₆ is hydrogen. In some embodiments, R₆ is C₁₋₂ alkyl substituted with di-C₁₋₆ alkylamine or 3 to 6-membered non-aromatic heterocyclic group containing at least a nitrogen.

In some embodiments, R₁ is C₁₋₃ alkyl, or C₁₋₃ alkyl substituted with C₄₋₃ alkoxy or 5- or 6-membered heterocyclic group having at least one selected from the group consisting of N, O and S. In some embodiments, R₁ is methyl.

In some embodiments, the compound is represent by Formula I-A,

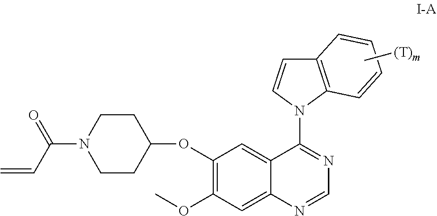

wherein T is a halogen and m is 1, 2 or 3.

In some embodiments, the compound is selected from the group consisting of

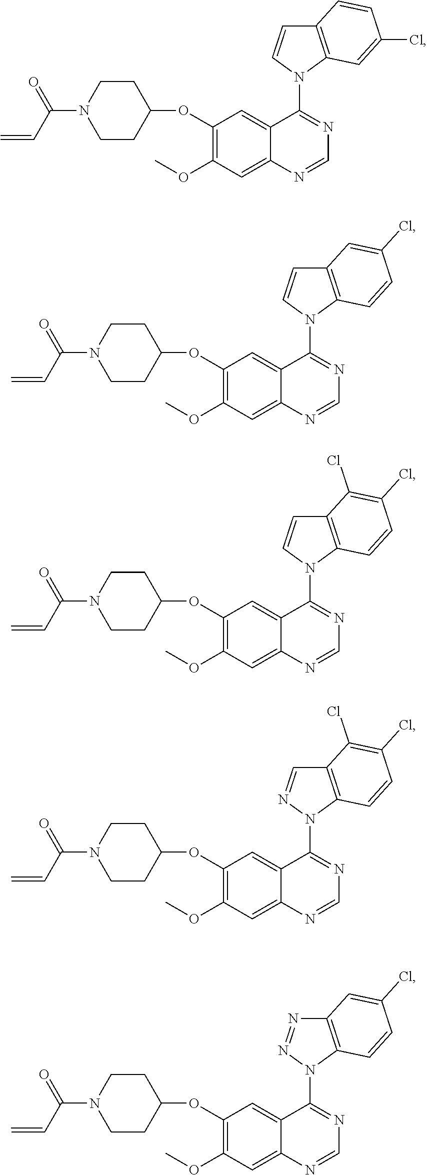 [SMILES: C=CC(=O)N1CCC(Oc2cc3c(-n4ccc5ccc(Cl)cc54)ncnc3cc2OC)CC1 ; C=CC(=O)N1CCC(Oc2cc3c(-n4ccc5cc(Cl)ccc54)ncnc3cc2OC)CC1 ; C=CC(=O)N1CCC(Oc2cc3c(-n4ccc5c(Cl)c(Cl)ccc54)ncnc3cc2OC)CC1 ; C=CC(=O)N1CCC(Oc2cc3c(-n4ncc5c(Cl)c(Cl)ccc54)ncnc3cc2OC)CC1 ; C=CC(=O)N1CCC(Oc2cc3c(-n4nnc5cc(Cl)ccc54)ncnc3cc2OC)CC1]

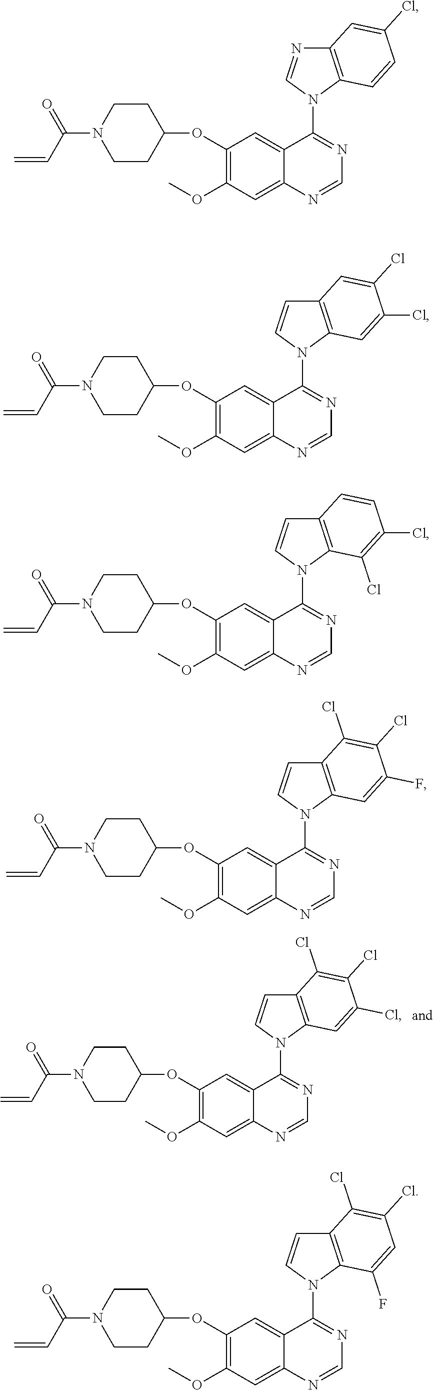

Another aspect of the disclosure provides a pharmaceutical composition comprising the compound or the pharmaceutically acceptable salt thereof described herein and one or more pharmaceutically acceptable carrier. In some embodiments, the pharmaceutical composition includes an additional cytotoxic agent selected from the group consisting of an antimetabolite, a mitotic inhibitor, alkylating agent, a platinum-based antineoplastic drug, an antibody-drug conjugate consisting of the EGFR monoclonal antibody and toxic payload such as T-DM1, a c-MET tyrosine kinase inhibitor, immune checkpoint inhibitors such as PD-1/PD-L1 or CTLA4, an mTOR inhibitor, a VEGF inhibitor, an aromatase inhibitor, a CDK4/6 inhibitor, and any combination thereof.

Another aspect of the disclosure provides a kit for treating cancer comprising the compound or a pharmaceutically acceptable salt thereof described herein, and an additional cytotoxic agent.

Another aspect of the disclosure provides a method of treating a cancer in a subject comprising administering to a subject in need thereof the compound or the pharmaceutical composition described herein. In some embodiments, the cancer is selected from the group consisting of non-small cell lung cancer, breast cancer, stomach cancer, colon cancer, pancreatic cancer, prostate cancer, myeloma, head and neck cancer, ovarian cancer, esophageal cancer, and metastatic cell carcinoma. In some embodiments, the cancer is characterized by having EGFR or HER2 mutations in exon 19 or exon 20.

### DETAILED DESCRIPTION

While the following text may reference or exemplify specific embodiments of a compound or a method of treating a disease or condition, it is not intended to limit the scope of the compound or method to such particular reference or examples. Various modifications may be made by those skilled in the art, in view of practical and economic considerations, such as the substituions of the compound and the amount or administration of the compound for treating or preventing a disease or condition.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “pharmaceutical composition” refers to a mixture of a compound disclosed herein with other chemical components, such as diluents or additional carriers. The pharmaceutical composition facilitates administration of the compound to an organism. Multiple techniques of administering a pharmaceutical composition exist in the art including, but not limited to, oral, injection, aerosol, parenteral, and topical administration. In some embodiments, pharmaceutically acceptable salts of the compounds disclosed herein are provided.

The term “carrier” refers to a chemical compound that facilitates the incorporation of a compound into cells or tissues.

The term “diluent” refers to chemical compounds diluted in water that will dissolve the composition of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound. As used herein, an “excipient” refers to an inert substance that is added to a composition to provide, without limitation, bulk, consistency, stability, binding ability, lubrication, disintegrating ability, etc., to the composition. A “diluent” is a type of excipient.

The term “physiologically acceptable” or “pharmaceutically acceptable” refers to a carrier or diluent that does not abrogate the biological activity and properties of the compound.

The term “therapeutically effective amount” refers to an amount of a compound effective to prevent, alleviate or ameliorate symptoms of disease or prolong the survival of the subject being treated. Determination of a therapeutically effective amount is well within the capability of those skilled in the art without undue experiments.

The term “alkyl” refers to monovalent or divalent saturated alkane radical groups particularly having up to about 18 carbon atoms, more particularly as a lower alkyl, from 1 to 8 carbon atoms and still more particularly, from 1 to 6 carbon atoms. The hydrocarbon chain may be either straight-chained or branched. The term “C1-C10 alkyl” refers to alkyl groups having 1, 2, 3, 4, 5, 6, 7, 8, 9 or 10 carbon atoms. Similarly, the term “C1-C6 alkyl” refers to alkyl groups having 1, 2, 3, 4, 5, or 6 carbon atoms. Non-limiting examples include groups such as methyl, ethyl, n-propyl, isopropyl, n-butyl, iso-butyl, tert-butyl, n-hexyl, n-octyl, tert-octyl and the like.

The term “cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like. The term “hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloheteroalkyl.

The term “halogen” refers to F, Cl, Br, or I.

The term “carboxamide” refers to a group of CONRR, wherein each R is independently a C1-C10 alkyl or an aryl.

The term “aromatic ring” or “aryl” refers to a monovalent or bivalent aromatic structure, which includes carbon rings where all ring atoms are carbons. The aromatic structure also includes heteroaromatic or heteroaryl rings where one or more ring atoms are heteratoms (e.g. oxygen, sulfur, nitrogen) or amino groups. Typical aryl groups having all carbon ring atoms include, but are not limited to, groups derived from aceanthrylene, acephenanthrylene, anthracene, azulene, benzene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, and the like. In some embodiments, an aryl group comprises from 6 to 14 carbon atoms.

Typical heteroaryl groups include, but are not limited to, groups derived from acridine, carbazole, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

The term “subject” or “patient” refers to a mammalian and includes humans and animals.

The term “treating” or “treatment” of any disease or condition refers, in some embodiments, to ameliorating the disease or disorder (i.e., arresting or reducing the development of the disease or at least one of the clinical symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

The term “EGFR” or “Epidermal growth factor receptor” or “EGFR” refers to a tyrosine kinase cell surface receptor including those encoded by one of four alternative transcripts appearing as GenBank accession NM_005228.3, NM .201282.1, NM_201283.1 and NM_201284.1. Variants of EGFR include an insertion in exon 20 or exon 19.

The term “HER2” refers to human epidermal growth factor receptor 2. Variants of HER2 include an insertion in exon 20 or exon 19.

This document discloses a novel class of quinozoline compounds which exhibit potent and selective inhibitivie activities against EGFR/HER2 targets. An aspect of the invention provides a compound of Formula I or a pharmaceutically acceptable salt thereof.

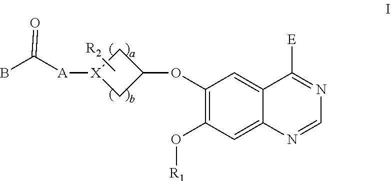

R₁ is hydrogen, C₁₋₆ alkyl, or C₁₋₆ alkyl substituted with C₁₋₆ alkoxy or 5- or 6-membered heterocyclic group having at least one selected from the group consisting of N, O and S. R₂ is hydrogen, —COOH, C₁₋₆ alkyloxycarbonyl, or amido N-unsubstituted or N-substituted with Y.

Y is hydroxy or C₁₋₆ alkyl or C₁₋₆ alkyl substituted with Z;

Z is hydroxy, Co₁₋₃ alkoxy, C₁₋₃ alkylthio, C₁₋₃ alkylsullbnyl, di-C₁₋₃alkylatnine, C₁₋₆ alkyl, phenyl or 5- or 6-membered aromatic or non-aromalic heterocyclic group, said heterocyclic group containing one to four of the moiety selected from the group consisting of N, O, S, SO, and SO₂ and said aryl and heterocyclic group being unsubstituted, or substituted with substituents selected from the group consisting of halogen, hydroxyl, amino, nitro, cyano, C₁₋₆-alkyl, C₂₋₆ alkenyl, C₁₋₆-alkynyl, C₁₋₆ alkoxy, C₁₋₆ monoalkylamino and C₁₋₆dialkylamino.

A is NH, or NC₁₋₆alkyl when X is CH; alternatively, A is void when X is N or NH.

B is

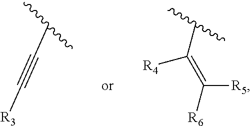

wherein: R₃, R₄, R₅ and R₆ are each independently selected from the group consisting of hydrogen, halogen, N—C₁₋₆alkyl or N-hydroxy amido or C—C₁₋₆ alkyl reverse amido(-NHCOC₁₋₆), hydroxycarbonyl (—COOH), C₁₋₆ alkyloxycarbonyl (—COOC₁₋₆), C₁₋₆ alkyl, and C₁₋₆ alkyl substituted with a hydroxy, di-C₁₋₆ alkylamine or 3 to 6-membered heterocyclic group haying at least one selected from the group consisting of N, O and S, wherein the 5- or 6-membered heterocyclic group is unsubstiiuted or substituted with C₁₋₄ alkyl.

E is selected from the group consisting of

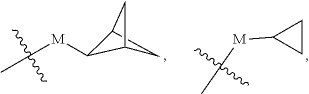

and 9 to 12 membered bicyclic ring, wherein each of these is optionally substituted with one or more substituents selected from the group consisting of halogen, hydroxy, cyano, nitro, (mono-, di-, or trihalogenoimethyl, mercapto, C₁₋₆ alkylthio, acrylamido, C₁₋₆alkyl, C₂₋₆ alkenyl, C₂₋₆ aikynyl, C₁₋₆ alkoxy, phenyloxy, and C₁₋₆ dialkylamino, further wherein M is selected from the group consisting of O, S, NH, NC₁₋₆ alkyl and C₁₋₆ alkyl.

The integer a and b are each an integer selected from 0, 1, 2, 3, 4, 5 and 6, with the proviso that a and b are not simultaneously 0. In some embodiments, R₂ is H and a is 1 or 2.

The compounds of formula I can be prepared by various linear or convergent synthetic approaches. For instance, the scheme below illustrates a general approach where two moieties are attached to a pre-assembled quinazoline core. Alternatively, the E moiety can be introduced to the bicyclic aromatic core through well-known chemistry such as Suzuki coupling reactions.

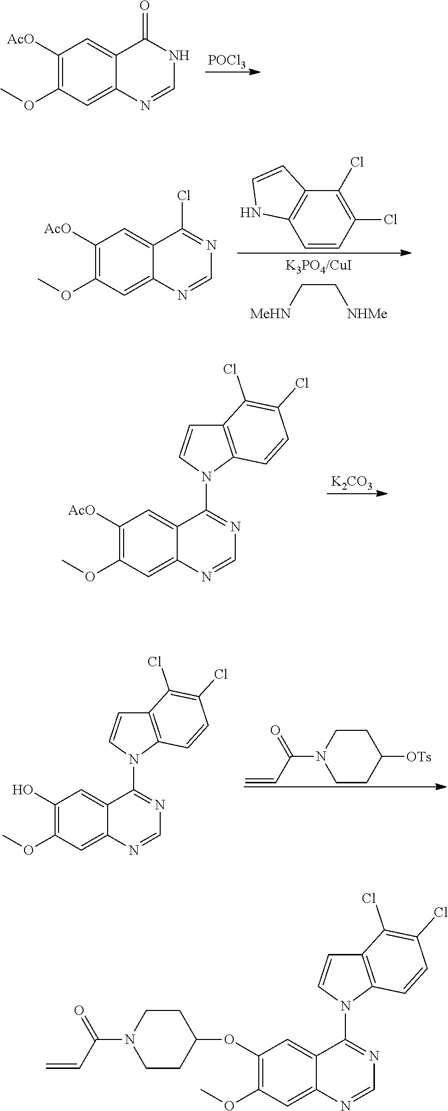

The E moiety can also be introduced to the quinazoline core via other types of coupling reaction such as Suzuki coupling and Stille coupling depending on the specific bicyclic ring structure. One of ordinary skill in the art can readily identify a suitable condition for synthesizing the target compound without undue experiments. Alternative approaches to compounds of Formula I can be developed based on those described in U.S. Pat. Nos. 9,518,043 and 8,859,767.

Another aspect of the present disclosure provides a pharmaceutical composition containing a therapeutically effective amount of the above described compound and a pharmaceutically acceptable carrier. In some embodiments, the pharmaceutical composition can futher include one or more additional cytotoxic agents. Non-limiting exampels of the additional agent include an antimetabolite, a mitotic inhibitor, alkylating agent, a platinum-based antineoplastic drug, an antibody-drug conjugate consisting of the EGFR monoclonal antibody and toxic payload such as T-DM1, a c-MET tyrosine kinase inhibitor, immune checkpoint inhibitors such as PD-1/PD-L1 or CTLA4, an mTOR inhibitor, a VEGF inhibitor, an aromatase inhibitor, a CDK4/6 inhibitor, and any combination thereof The pharmaceutical composition may also contain one or more physiologically acceptable surface active agents, additional carriers, diluents, excipients, smoothing agents, suspension agents, film forming substances, and coating assistants, or a combination thereof; and a composition disclosed herein. Acceptable additional carriers or diluents for therapeutic use are well known in the pharmaceutical art, and are described, for example, in Remington's Pharmaceutical Sciences, 18th Ed., Mack Publishing Co., Easton, PA (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, microcrystalline cellulose, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents. The pharmaceutical compounds described herein can be administered to a human patient per se, or in pharmaceutical compositions where they are mixed with other active ingredient(s), as in combination therapy, or suitable carriers or excipient(s). In some embodiments, a dosage form includes those forms in which the compound is admistered per se. In addition, a dosage form may include a pharmaceutical composition. In any case, the dosage form may comprise a sufficient amount of the compound to treat a cancer as part of a particular administration protocol, as would be understood by those of skill in the art. Techniques for formulation and administration of the compounds of the instant application may be found in “Remington's Pharmaceutical Sciences,” Mack Publishing Co., Easton, PA, 18th edition, 1990.

Suitable routes of administration may, for example, include oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The compound can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions may be manufactured in a manner that is itself known, e.g., by means of conventional mixing, dissolving, granulating, dragee-making, levigating, emulsifying, encapsulating, entrapping or tabletting processes.

Pharmaceutical compositions may be formulated in any conventional manner using one or more physiologically acceptable carriers comprising excipients and auxiliaries which facilitate processing of the active compounds into preparations which can be used pharmaceutically. Proper formulation is dependent upon the route of administration chosen.

Any of the well-known techniques, diluents, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Aqueous injection suspensions may contain substances which increase the viscosity of the suspension, such as sodium carboxymethyl cellulose, sorbitol, or dextran.

Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the composition can be formulated readily by combining the compositions of interest with pharmaceutically acceptable carriers well known in the art. Such carriers, which may be used in addition to the cationic polymeric carrier, enable the compositions to be formulated as tablets, pills, dragees, capsules, liquids, gels, syrups, slurries, suspensions and the like, for oral ingestion by a patient to be treated. Pharmaceutical preparations for oral use can be obtained by combining the active compound with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxyprop ylmethyl-cellulo se, sodium carboxymethylcellulose, and/or polyvinylpyrrolidone (PVP), e.g., Povidone. If desired, disintegrating agents may be added, such as the cross-linked polyvinylpyrrolidone (e.g. Crospovidone), agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in a conventional manner Administration to the buccal mucosa and sublingually are contemplated.

For administration by inhalation, the composition can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions may also be formulated in rectal compositions such as suppositories or retention enemas, e.g., containing conventional suppository bases such as cocoa butter or other glycerides.

Another aspect of this disclosure provides a kit for treating cancer comprising the compound or the pharmaceutically acceptable salt thereof disclosed herein, and an additional cytotoxic agent. The cytotoxic agent is as described above. Another aspect of this disclosure provides a method of treating a cancer in a subject comprising administering to the subject in need a therapeutically effective amount of a compound of Formula I, a pharmaceutically salt thereof, or a pharmaceutical composition thereof. Specific embodiments of the compound of Formula I, its salt or pharmaceutical composition are as described above. Non-limiting examples of the cancer is selected from the group consisting of non-small cell lung cancer, breast cancer, stomach cancer, colon cancer, pancreatic cancer, prostate cancer, myeloma, head and neck cancer, ovarian cancer, esophageal cancer, and metastatic cell carcinoma.

Certain embodiments of the present disclosure concern determining if a subject has one or more EGFR and/or HER2 exon 20 mutations, such as an insertion mutation. The subject may have 2, 3, 4, or more EGFR exon 20 mutations and/or HER2 exon 20 mutations. Mutation detection methods are known the art including PCR analyses and nucleic acid sequencing as well as FISH and CGH. In particular aspects, the exon 20 mutations are detected by DNA sequencing, such as from a tumor or circulating free DNA from plasma.

The EGFR exon 20 mutation(s) may comprise one or more point mutations, insertions, and/or deletions of 3-18 nucleotides between amino acids 763-778. The one or more EGFR exon 20 mutations may be located at one or more residues selected from the group consisting of A763, A767, S768, V769, D770, N771, P772, and H773. In another embodiment, patients may have one or more classical EGFR mutations exon 19 mutation (exon 19 deletion, L858R, and L861Q).

EGFR exon 20 insertions may include H773_V774insH, A767_v769ASV, N771_P772insH, D770_N771insG, H779_V774insH, N771delinsHH, S768_D770dupDVD, A767_V769dupASV, A767_V769dupASV, P772_H773dup, N771_H773dupNPH, S768_D770dupSVD, N771 delinsGY, S768_D770delinsSVD, D770_D770delinsGY, A767_V769dupASV, and/or H773dup. In particular aspects, the exon 20 mutations are A763insFQEA, A767insASV, S768dupSVD, V769insASV, D770insSVD, D770insNPG, H773insNPH, N771del insGY, N771del insFH and/or N771dupNPH.

In some aspects, the subject may have or develop a mutation at EGFR residue C797 which may result in resistance to the TKI, such as poziotinib. Thus, in certain aspects, the subject is determined to not have a mutation at EGFR C797.

The HER2 exon 20 mutation may comprise one or more point mutations, insertions, and/or deletions of 3-18 nucleotides between amino acids 770-785. The one or more HER2 exon 20 mutations may be at residue A775, G776, 5779, and/or P780. The one or more HER2 exon 20 mutations may be A775insV G776C, A775insYVMA, G776V, G776C V777insV, G776C V777insC, G776del insVV, G776del insVC, and/or P780insGSP.

In another embodiment, patients may have classical EGFR mutations such as exon 19 deletion, L858R, and L861Q.

In another embodiment, pateints with the EGFR exon 19 or 20 mutation suffer from a cancer selected from the group consisting of non-small cell lung cancer (NSCLC), breast cancer, stomach cancer, colon cancer, pancreatic cancer, prostate cancer, myeloma, head and neck cancer, ovarian cancer, esophageal cancer, and metastatic cell carcinoma. In one embodiment, the patients in need of the treatment is suffering from EGFR mutant NSCLC with an in-frame insertion within exon 20 of EGFR.

In at least one embodiment, methods of treating a patient suffering from a cancer having an EGFR mutant with an in-frame insertion within exon 20 of EGFR or a deletion within exon 19.

In some embodiments, the subject to be treated is a mammal, e.g., a primate, preferably a higher primate, e.g., a human (e g , a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy.

Alternatively, or in combination, the subject is, or is at risk of being, immunocompromised as a result of an infection.

Certain embodiments concern the administration of a composition of formula I to a subject determined to have EGFR or HER2 exon 20 mutation, such as an exon 20 insertion. In other embodiments, methods of treating a patient suffering from NSCLC with EGFR and HER 2 exon 20 mutation are described by administering the compound of formulat I or the pharmaceutically acceptable salt thereof, wherein E is the 5,5-, 5,6- or 6,6-bicyclic ring system containing up to three hetero atoms selected from oxygen, nitrogen or sulfur and preferably selected from the group consisting of naphthyridine, indole, benzoimidazole, benzotriazole, benzodioxaole, furopyridine, isoindole, pyridooxazine, pyrrolopyridine, quinoxaline, quinazoline, quinoline, isoquinoline, indazole, [1,2,4]triazolol1,5-alpyridine, 1,2,3,4-tetrahydroisoquinoline, 1,3-benzodioxole, 1-benzothiophene, 1H-indazole, 1H-pyrrolol[2,3-b]yridine, 1H-pyrrolo [2,3-c]pyridine, 1H-pyrrolo[3,2-b]pyridine, 1H-pyrrolo [3,2-b]pyridine, 2,1,3-benzoxadiazole, 3,4-dihydro-2H-pyrido[3,2-b][1,4]oxazine, 3H-imidazo[4,5-b]pyridine, 4,5,6,7-tetrahydropyrazolo[1,5-a]pyridine, furo[2,3-c]pyridine, furo[3,2-b]pyridine, imidazo[1,2-a]pyridine, and thieno[3,2-c]pyridin-4(5H)-one.

In some embodiments, the method further includes administering an additional cytotoxic agent. The cytotoxic agent is as described above.

In certain embodiments, the method of treatment of patients suffering from NSCLC further comprising an additional cytotoxic agent selected from the group consisting of an antimetabolite, a mitotic inhibitor, alkylating agent, a platinum-based antineoplastic drug, an antibody-drug conjugate consisting of the EGFR monoclonal antibody and toxic payload such as T-DM1, a c-MET tyrosine kinase inhibitor, immune checkpoint inhibitors such as PD-1/PD-L1 or CTLA4, an mTOR inhibitor, a VEGF inhibitor, an aromatase inhibitor, a CDK4/6 inhibitor, and any combination thereof.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively dosages may be based and calculated upon the surface area of the patient, as understood by those of skill in the art. The route of administration and dosage for the pharmaceutical compositions can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). In some embodiments, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some conditions, those same dosages, or dosages that are about 0.1% to about 500%, more preferably about 25% to about 250% of the established human dosage may be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine. Although the exact dosage will be determined on a drug-by-drug basis, in most cases, some generalizations regarding the dosage can be made. The daily dosage regimen for an adult human patient may be, for example, an oral dose of about 0.1 mg to 2000 mg of the active ingredient, preferably about 1 mg to about 500 mg, e.g. 5 to 200 mg. In other embodiments, an intravenous, subcutaneous, or intramuscular dose of the active ingredient of about 0.01 mg to about 100 mg, preferably about 0.1 mg to about 60 mg, e.g. about 1 to about 40 mg is used.

In cases of administration of a pharmaceutically acceptable salt, dosages may be calculated as the free acid. In some embodiments, the composition is administered 1 to 4 times per day. Alternatively, the compositions may be administered by continuous intravenous infusion, preferably at a dose of up to about 1000 mg per day. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections. In some embodiments, the compounds will be administered for a period of continuous therapy, for example for a week or more, or for months or years.

Dosage amount and interval may be adjusted individually to provide plasma levels of the active moiety which are sufficient to maintain the antitumor effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered may be dependent on the subject being treated, on the subject's weight, the severity of the condition, the manner of administration and the judgment of the prescribing physician.

Compositions disclosed herein can be evaluated for efficacy and toxicity using known methods. For example, the toxicology of the compound may be established by determining in vitro toxicity towards a cell line, such as a mammalian, and preferably human, cell line. The results of such studies are often predictive of toxicity in animals, such as mammals, or more specifically, humans Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound formulated in a compatible pharmaceutical carrier may also be prepared, placed in an appropriate container, and labeled for treatment of an indicated condition.

### EXAMPLE

The results of modeling studies on the Poziotinib and compound A-1 are shown in Table 1. The calculated values for nonbonding interaction energy indicate that Compound A-1 is more selective against EGFR mutant than Poziotinib.

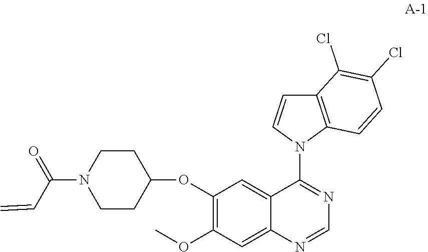 [SMILES: C=CC(=O)N1CCC(Oc2cc3c(-n4ccc5c(Cl)c(Cl)ccc54)ncnc3cc2OC)CC1]

TABLE 1
Modeling Study for the Nonbonding Interaction Energy, Strain
and Presence of a Hydrogen Bond with the Kinase Hinge

| Agent | Target | Non-bonding | Strain | Selectivity |
|---|---|---|---|---|
| Poziotinib | EGFR WT | −46.2 | 6.5 | |
| | EGFR mutant | −48.8 | 11.6 | No selectivity |
| A-1 | EGFR WT | −42.6 | 13.8 | |
| | EGFR mutant | −60.2 | 14.9 | Mutant select |

It will be appreciated by persons skilled in the art that fibers described herein are not limited to what has been particularly shown and described. Rather, the scope of the fiber is defined by the claims which follow. It should further be understood that the above description is only representative of illustrative examples of embodiments. The description has not attempted to exhaustively enumerate all possible variations. The alternate embodiments may not have been presented for a specific portion of the fiber, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

1. A compound or a pharmaceutically acceptable salt thereof, wherein the compound is represented by Formula I: 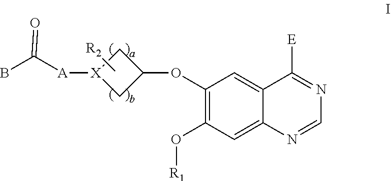
wherein:
R₁ is hydrogen, C₁₋₆ alkyl, or C₁₋₆ alkyl substituted with C₁₋₆ alkoxy or 5- or 6-membered heterocyclic group having at least one selected from the group consisting of N, O and S;
R₂ is hydrogen, —COOH, C₁₋₆ alkyloxycarbonyl, or amido N-unsubstituted or N-substituted with Y;
Y is hydroxy or C₁₋₆ alkyl or C₁₋₆ alkyl substituted with Z;
Z is hydroxy, C₁₋₃ alkoxy, C₁₋₃ alkylthio, C₁₋₃ alkyl sulfonyl, di-C₁₋₃alkylamine, C₁₋₆ alkyl, phenyl or 5- or 6-membered aromatic or non-aromatic heterocyclic group, said heterocyclic group containing one to four of the moiety selected from the group consisting of N, O, S, SO, and SO₂ and said aryl and heterocyclic group being unsubstituted, or substituted with substituents selected from the group consisting of halogen, hydroxyl, amino, nitro, cyano, C₁₋₆alkyl, C₂₋₆ alkenyl, C₂₋₆ alkynyl, C₁₋₆ alkoxy, C₁₋₆ monoalkylamino and C₁₋₆ dialkylamino. A is NH, or NC₁₋₆alkyl when X is CH; alternatively, A is void when X is N or NH;
B is 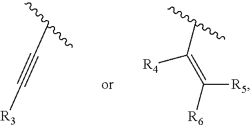
wherein:
R₃, R₄, R₅ and R₆ are each independently selected from the group consisting of hydrogen, halogen,
N—C₁₋₆alkyl or N-hydroxy amido or C—C₁₋₆ alkyl reverse amido(—NHCOC₁₋₆), hydroxycarbonyl (—COOH), C₁₋₆ alkyloxycarbonyl (—COOC₁₋₆), C₁₋₆ alkyl, and C₁₋₆ alkyl substituted with a hydroxy, di-C₁₋₆ alkylamine or 3 to 6-membered heterocyclic group having at least one selected from the group consisting of N, O and S, wherein the 5- or 6-membered heterocyclic group is unsubstituted or substituted with C₁₋₄ alkyl;
E is selected from the group consisting of 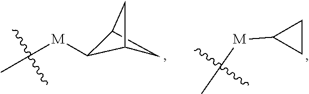
and 9 to 12 membered bicyclic ring,
wherein each of these is optionally substituted with one or more substituents selected from the group consisting of halogen, hydroxy, cyano, nitro, (mono-, di-, or trihalogeno)methyl, mercapto, C₁₋₆ alkylthio, acrylamido, C₁₋₆ alkyl, C₂₋₆ alkenyl, C₂₋₆ alkynyl, C₁₋₆ alkoxy, phenyloxy, and C₁₋₆ dialkylamino, further wherein M is selected from the group consisting of O, S, NH, NC₁₋₆ alkyl and C₁₋₆ alkyl;
and
a and b are each an integer ranging from 0 to 6, with the proviso that a and b are not simultaneously 0.

2. The compound or the pharmaceutically acceptable salt thereof of claim 1, wherein E is 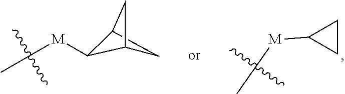
each of which is optionally substituted to one to three halogens; and
M is NH.

3. The compound or the pharmaceutically acceptable salt thereof of claim 1, wherein E is the bicyclic ring selected from the group consisting of naphthyridine, indole, benzoimidazole, benzotriazole, benzodioxaole, furopyridine, isoindole, pyridooxazine, pyrrolopyridine, quinoxaline, quinazoline, quinoline, isoquinoline, indazole, [1,2,4]triazolo[1,5-a]pyridine, 1,2,3,4-tetrahydroisoquinoline, 1,3-benzodioxole, 1-benzothiophene, 1H-indazole, 1H-pyrrolo[2,3-b]pyridine, 1H-pyrrolo[2,3-c]pyridine, 1H-pyrrolo[3,2-b]pyridine, 1H-pyrrolo[3,2-c]pyridine, 2,1,3-benzoxadiazole, 3,4-dihydro-2H-pyrido[3,2-b][1,4]oxazine, 3H-imidazo[4,5-b]pyridine, 4,5,6,7-tetrahydropyrazolo[1,5-a]pyridine, furo[2,3-c]pyridine, furo[3,2-b]pyridine, imidazo[1,2-a]pyridine, and thieno[3,2-c]pyridin-4(5H)-one; wherein each is optionally substituted with one or more substituents selected from the group consisting of halogen, hydroxy, cyano, nitro, (mono-, di-. or trihalogeno)methyl, mercapto, C₁₋₆ alkylthio, acrylamido, C₁₋₆ alkyl, C₂₋₆ alkenyl, C₂₋₆ alkynyl, C₁₋₆ alkoxy, phenyloxy, and C₁₋₆ dialkylaniino.

4. The compound or the pharmaceutically acceptable salt thereof of claim 1, wherein E is the optionally substituted bicyclic ring selected from the group consisting of naphthyridine, indole, benzoimidazole, benzotriazole, isoindole, quinoxaline, quinazoline, quinoline, isoquinoline, and indazole.

5. The compound or the pharmaceutically acceptable salt thereof of claim 1, wherein E is the optionally substituted bicyclic ring selected from the group consisting of naphthyridine, indole, benzoimidazole, benzotriazole, and indazole.

6. The compound or the pharmaceutically acceptable salt thereof of claim 1, wherein E is the optionally substituted bicyclic ring selected from the group consisting of

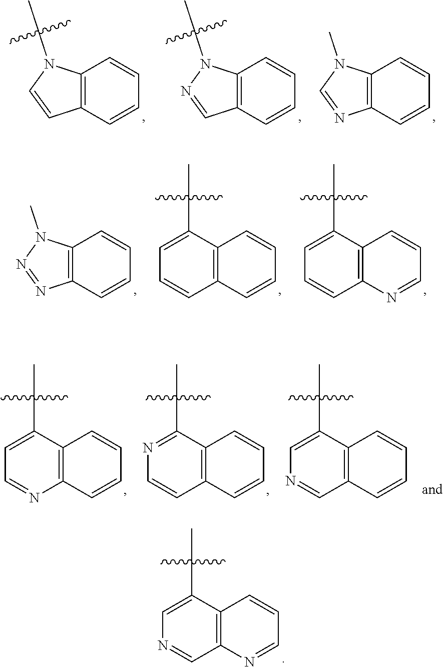

7. The compound or the pharmaceutically acceptable salt thereof of claim 1, wherein E is selected from the group consisting of

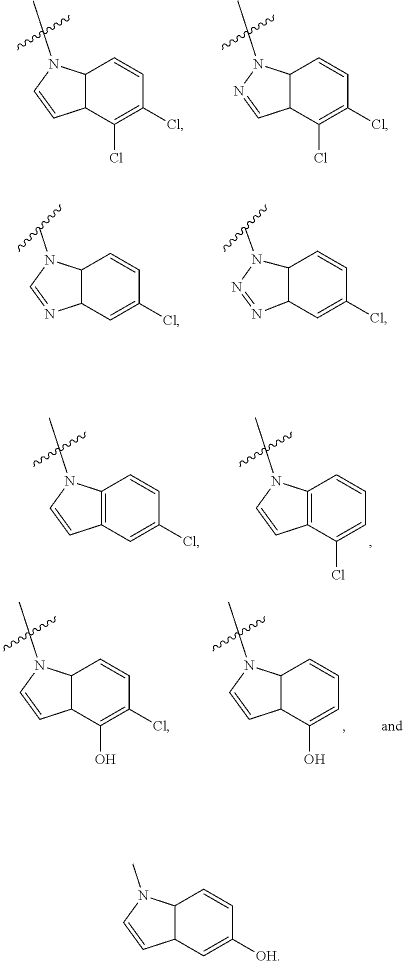

8. The compound or the pharmaceutically acceptable salt thereof of claim 1, wherein 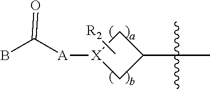
is selected from the group consisting of

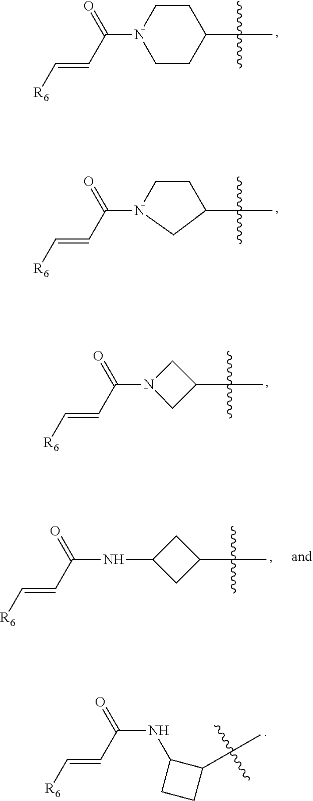

9. The compound or the pharmaceutically acceptable salt thereof of claim 8, wherein R₆ is hydrogen.

10. The compound or the pharmaceutically acceptable salt thereof of claim 8, wherein R₆ is C₁₋₂ alkyl substituted with di-C₁₋₆ alkylamine or 3 to 6-membered non-aromatie heterocyclic group containing at least a nitrogen.

11. The compound or the pharmaceutically acceptable salt thereof of claim 1, wherein R₁ is C₁₋₃ alkyl, or C₁₋₃ alkyl substituted with C₁₋₃ alkoxy or 5- or 6-rnembered heterocyclic group having at least one selected from the group consisting of N, O and S.

12. The compound or the pharmaceutically acceptable salt thereof of claim 1, wherein is Methyl.

13. The compound or the pharmaceutically acceptable salt thereof of claim 1, wherein the compound is represented by Formula I-A, 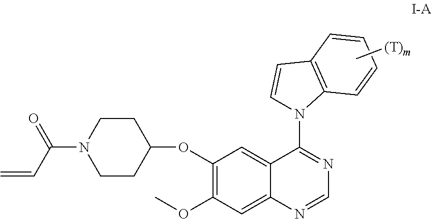
wherein T is a halogen and m is 1, 2 or 3.

14. The compound or the pharmaceutically acceptable salt thereof of claim 1, wherein the compound is selected from the group consisting of

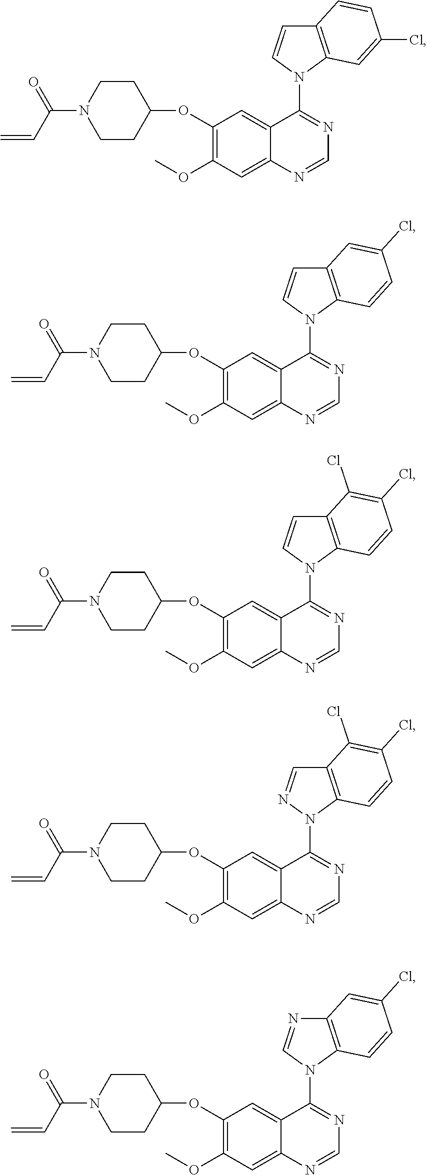 [SMILES: C=CC(=O)N1CCC(Oc2cc3c(-n4ccc5ccc(Cl)cc54)ncnc3cc2OC)CC1 ; C=CC(=O)N1CCC(Oc2cc3c(-n4ccc5cc(Cl)ccc54)ncnc3cc2OC)CC1 ; C=CC(=O)N1CCC(Oc2cc3c(-n4ccc5c(Cl)c(Cl)ccc54)ncnc3cc2OC)CC1 ; C=CC(=O)N1CCC(Oc2cc3c(-n4ncc5c(Cl)c(Cl)ccc54)ncnc3cc2OC)CC1 ; C=CC(=O)N1CCC(Oc2cc3c(-n4cnc5cc(Cl)ccc54)ncnc3cc2OC)CC1]

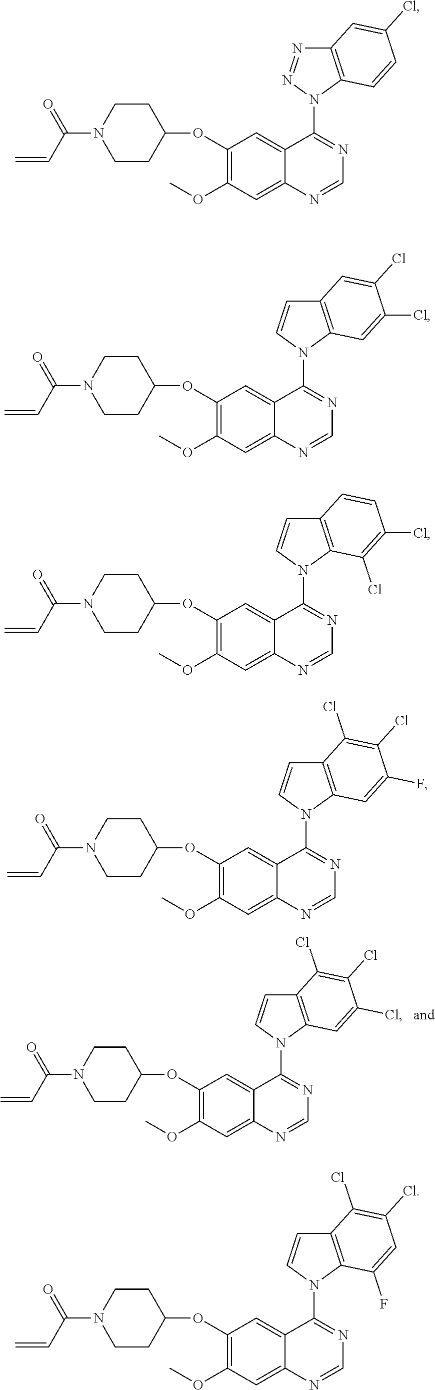

15. A pharmaceutical composition comprising the compound or the pharmaceutically acceptable salt thereof of claim 1 and one or more pharmaceutically acceptable carrier.

16. The pharmaceutical composition of claim 15, further comprising an additional cytotoxic agent selected from the group consisting of an antimetabolite, a mitotic inhibitor, alkylating agent, a platinum-based antineoplastic drug, an antibody-drug conjugate consisting of the EGFR monoclonal antibody and toxic payload such as T-DM1, a c-MET tyrosine kinase inhibitor, immune checkpoint inhibitors such as PD-1/PD-L1 or CTLA4, an mTOR inhibitor, a VEGF inhibitor, an aromatase inhibitor, a CDK4/6 inhibitor, and any combination thereof.

17. A kit for treating cancer comprising the compound or the pharmaceutically acceptable salt thereof of claim 1, and an additional cytotoxic agent.

18. The kit of claim 17, wherein the cancer is characterized by having EGFR or HER₂ mutations in exon 19 or exon 20.

19. The kit of claim 17, wherein the additional cytotoxic agent selected from the group consisting of an antimetabolite, a mitotic inhibitor, alkylating agent, a platinum-based antineoplastic drug, an antibody-drug conjugate consisting of the EGFR monoclonal antibody and toxic payload such as T-DM1, a c-MET tyrosine kinase inhibitor, immune checkpoint inhibitors such as PD-1/PD-L1 or CTLA4, an mTOR inhibitor, a VEGF inhibitor, an aromatase inhibitor, a CDK4/6 inhibitor, and any combination thereof.

20. A method of treating a cancer in a subject comprising administering to a subject in need thereof the compound of claim 1.

21. The method of claim 20, wherein the cancer is selected from the group consisting of non-small cell lung cancer, breast cancer, stomach cancer, colon cancer, pancreatic cancer, prostate cancer, myeloma, head and neck cancer, ovarian cancer, esophageal cancer, and metastatic cell carcinoma.

22. The method of claim 20, wherein the cancer is characterized by having EGFR or HER₂ mutations in exon 19 or exon 20.
